(19) United States
(12) Patent Application Publication
(10) Pub. No.: US 2005/0118660 A1
(43) Pub. Date: Jun. 2, 2005

(54) Method and kit for identifying pseudomonas aeruginosa

(76) Inventor: Carmen V. Sciortino Jr, Goshen, KY (US)

Correspondence Address: Law Office Dinesh Agarwal, Suite 330, 5350 Shawnee Road, Alexandria, VA, 22312, US

(21) Appl. No.: 10/502,464

(22) PCT Filed: Mar. 19, 2003
(86) PCT No.: PCT/US03/06715

Related U.S. Application Data
(60) Provisional application No. 60/365,812, filed on Mar. 21, 2002.

(51) Int. Cl. G01N 33/554; G01N 33/569

(52) U.S. Cl. 435/7.32; 530/388.4

(57) ABSTRACT

A method of identifying a bacteria, such as Pseudomonas aeruginosa, in a sample, includes providing a sample suspect of comprising a bacteria to be identified, exposing the sample to an antibody specific for a lipoprotein of the bacteria and an agglutination reagent, allowing the sample to react with the antibody and the agglutination reagent, wherein the presence of the bacteria is indicated if an agglutination occurs. A kit for testing the presence of a bacteria, such as Pseudomonas aeruginosa, includes an agglutination reagent and an antibody specific for a lipoprotein of the bacteria.

### CROSS-REFERENCE TO RELATED APPLICATIONS

The present application claims priority on prior U.S. Provisional Application Ser. No. 60/365,812, filed Mar. 21, 2002, and which is incorporated herein in its entirety by reference.

### BACKGROUND OF THE INVENTION

The present invention is generally directed to identifying a bacteria in a sample, and more particularly to a method and kit for identifying or assaying Pseudomonas aeruginosa.

Pseudomonas aeruginosa is an oxidase-positive, gram-negative, rod-shaped organism that is found ubiquitously in the environment. It is versatile in its habitat and can grow in soil, water, and on plant and animal tissue. It is an opportunistic organism and one of the most problematic nosocomial pathogens capable of causing disease in susceptible individuals such as people who have cystic fibrosis, cancer, burns, or some immune system deficiency (Jaffe, Lane, and Bates 2001). Case fatality can be as high as 50 percent due to a combination of weakened host defenses, bacterial resistance to antibiotics, and the production of extra cellular-bacterial enzymes and toxins (Iglewski 2002). P. aeruginosa often colonizes hospital food, sinks, taps, mops, and respiratory equipment. The infection is spread from patient to patient via contact with fomites or by ingestion of contaminated food and water (Iglewski 2002).

P. aeruginosa is clinically indistinguishable from other gram-negative bacteria that also cause these sorts of infections but that have a lower morbidity and mortality rate. Therefore, early and accurate diagnosis is important. This is particularly important as P. aeruginosa is well known for being resistant to a wide spectrum of antibiotics.

Healthy individuals who come into contact with people with P. aeruginosa infections are not at risk of developing the infection themselves (Pseudomonas Genome Project 2002). In fact, P. aeruginosa is a resident of the intestinal tract in about 10 percent of healthy individuals, and is found sporadically in moist areas of the human skin and in the saliva (Chamberlain 2002).

The major clinical features used in diagnosis in situ are pus formation, pyocyanin formation in about 90 percent of cases, and fluorescein formation which can be viewed in the dark with a Wood's UV light for fluorescence. There are 13 antigenic groups of P. aeruginosa, which in the future, may be treated differentially with immunotherapy (Chamberlain 2002). This bacterium can be distinguished from other pseudomonad strains by growth at 42° C. (Washington State University 2002).

P. aeruginosa is also the most important human pathogen in the genus Pseudomonas (Kiska and Gilligan, 1999). It can also cause superficial skin infections, osteomyelitis, sepsis, folliculitis, swimmer's ear, endocarditis in intravenous-drug users, urinary tract infections, otitis media, and contact lens associated corneal ulcer (Dini et al., 2000; Pollack, 2000). As noted above, P. aeruginosa is also a major cause of nosocomial infection (Emori and Gaynes, 1993). In most clinical microbiology laboratories, P. aeruginosa is one of the top three clinical isolates, therefore its frequency of isolation is remarkable. Since P. aeruginosa is also highly antibiotic resistant, specific classes of antibiotics are recommended for their anti-pseudomonas activity. Therefore, a 2448 hour delay in organism identification may delay appropriate therapy and adversely affect patient morbidity.

Few individuals outside of the clinical microbiology laboratory setting may realize the difficulty that automated technology has with the identification of Pseudomonas aeruginosa. Although automated instruments are advertised as rapid, the best instruments require at least 18 hours to identify P. aeruginosa. In contrast, the same instrument can identify E. coli in 4 hours. Since a 24 hour pre-growth of the organism is needed, the total average turn-around-time (TAT) for the identification of P. aeruginosa is 46-48 hours. Before 1980, the same was true for the identification of Staphylococcus aureus, until a rapid latex agglutination test was developed. Now most microbiology laboratories can identify S. aureus in 14-18 hours using one of the many rapid tests available. Unfortunately, there has not been forthcoming a rapid test for P. aeruginosa until now. The majority (80%) of bacteriological isolates in a microbiology laboratory are one or more of Staph aureus, P. aeruginosa, and Escherichia coli. Therefore, there is a great need in the routine microbiology laboratory for a cost effective rapid test for P. aeruginosa.

In 1988, the inventor of the present invention contributed to the development of a monoclonal antibody (MAB PS2) which recognized a 8 KDa outer membrane protein which was later identified as lipoprotein I (LP I) (Sciortino 1993). LP I was detected by dot blot analysis in the outer membrane of 99.3% of P. aeruginosa strains (n=300). Studies were performed comparing the predictive value of MAB PS2 to the biochemical characterization of Pseudomonas aeruginosa by two automated instruments, the Vitek Automated Microbiology System (AMS), Sensititre, and a semi-automated instrument, the Dynatech 2000 (Sciortino et al. 1987). The evaluation showed an 89.7% agreement on organism identification between instruments. Discrepant results between MAB PS2 and the Dynatech 2000, the Sensititre, or the AMS were checked by conventional tubed media identification schemes or the VAMC reference laboratory. In the screening of clinical isolates reported as P. aeruginosa by the Dynatech 2000, MAB PS2 negatively reacted with two organisms which were later determined by conventional tests not to be P. aeruginosa since they did not grow on centrimide or at 42° C.

In screening several stock cultures of gram-negative organisms, two strains which were previously unidentified and listed only by a catalog number gave positive reactions with PS2. These strains grew on centrimide and at 42° C. which is indicative of P. aeruginosa. When tested by the AMS, one strain was identified as P. aeruginosa with 99% confidence while the other strain only showed a 14% probability of being P. aeruginosa. Because this organism grew on centrimide and at 42° C., it was correctly identifed by MAB PS2 as P. aeruginosa. This indicates that the MAB PS2 can be more sensitive in the recognition of this organism than the AMS.

Recent studies have involved development of test reagents and definition of specific reactivities of the molecules. For instance, the inventor discovered that P. aeruginosa (LP I) expression is time dependent. Twenty-four hour cultures express very little LP I in the outer membrane whereas 48 h cultures have large amounts of LP I associated with the outer membrane. It then became necessary to extract LP I from the inner membrane for maximum antibody reactivity at 24 h. Another problematic technical difficulty in the past studies involved adjusting trace metal concentrations. Too much magnesium caused auto-agglutination of bacteria with the reagents although Mg++ was required to release LP I from the bacterial membrane complex. Therefore, recent studies have focused on optimization of the co-agglutination test and less on the sensitivity and specificity of the test.

Diagnosis of P. aeruginosa therefore, depends on its isolation and laboratory identification. It can be cultured on most general-purpose media and is commonly isolated on blood agar plates or eosin-methylthionine blue agar. It is identified on the basis of its gram morphology, inability to ferment lactose, a positive oxidase reaction, its fruity odor, and its ability to grow at 42° C. It fluoresces under ultraviolet radiation and this is useful in suggesting its presence in wounds (Iglewski 2002).

The current automated technology for identification of P. aeruginosa is Vitek Automated Microbiology System. This method costs approximately $7 per sample (Jaffe, Lane, and Bates, 2001).

Biotec Laboratories Limited, a U.K. biotechnology company, is focusing on a novel technology termed phage amplification that is based on the presence of a specific bacteriophage for identifying the presence of the target bacteria within a 4-hour period (Stewart et al. 1998). Biotec's phage amplification is a platform technology with a test for P. aeruginosa in the research and development phase (per personal communication). The company claims this technology will have the advantages of speed, accuracy, simplicity, and low cost (Biotec Laboratories Limited 2002).

Jaffe, Lane, and Bates (2001) reportedly have developed a rapid DNA extraction and polymerase chain reaction (PCR) method for identifying P. aeruginosa directly from clinical samples including blood, sputum, and urine. Two versions of this test have been developed: standard PCR and rapid-cycle PCR, with the former having a turnaround time of 4 hours and the latter of just 1 hour. The results obtained using this technique are 100 percent in concordance with those achieved using the conventional culture methods. They estimate the direct supply cost of the DNA extraction and PCR to be $2.22 per sample. It is important to note, however, that the equipment costs involved in real-time PCR technology are considerable and have almost certainly not been factored into this calculation. This work has been accomplished in collaboration with Commonwealth Biotechnologies, Inc. (Virginia), a contract research organization. The developers do not appear to be selling this kit, nor does Commonwealth Biotechnologies provide any further information about it on the company's web site (Commonwealth Biotechnologies, Inc. 2002).

Therefore, there has not yet been a rapid test for P. aeruginosa that would allow its identification on the first day of culture. Automated microbiology instruments still require 18-24 h to identify Pseudomonas. Roche Inc, has recently introduced a rapid light-cycler test for rapid identification of P. aeruginosa, but equipment cost and test complexity may limit its use in the routine clinical laboratory.

In view of the above, there is a need in the industry for a rapid, accurate, reliable and inexpensive test for the identification of Pseudomonas aeruginosa.

### OBJECTS AND SUMMARY OF THE INVENTION

The principal object of the present invention is to provide a method and kit for identifying Pseudomonas aeruginosa which overcomes the drawbacks associated with conventional techniques and materials.

An object of the present invention is to provide a method and kit for identifying Pseudomonas aeruginosa which is fast, reliable, accurate, sensitive, specific, and inexpensive.

Another object of the present invention is to provide a method and kit for identifying Pseudomonas aeruginosa which significantly reduces the test turnaround time to about 18-20 hours, and is a single assay as opposed to a battery of tests required by the conventional methods. In particular, the instant invention reduces the laboratory analysis turnaround time by about 24 hours, providing diagnosis the day after the sample is received and consequently allowing antibiotic or other suitable treatment to begin a day earlier. This is significant given the morbidity associated with this infection. The test has a sensitivity of about 99.3% and a specificity of about 95%.

Yet another object of the present invention is to provide a method and kit for identifying Pseudomonas aeruginosa which uses an anti body that is specific for a lipoprotein (LP1) on the surface of Pseudomonas aeruginosa, and an agglutination reagent to indicate a positive result.

A further object of the present invention is to provide a method and kit for identifying Pseudomonas aeruginosa which costs significantly less than the conventional tests.

Yet a further object of the present invention is to provide a method and kit for identifying Pseudomonas aeruginosa which is more likely to be accepted by the clinical diagnostics community because it is an antibody-based diagnostic test for bacterial infections than the conventional tests based on other formats, such as DNA diagnostics.

Still a further object of the present invention is to provide a method and kit for identifying Pseudomonas aeruginosa which would fill a market need for a rapid and reliable test for a problematic pathogen that causes significant morbidity and mortality.

An additional object of the present invention is to provide a method and kit for identifying Pseudomonas aeruginosa which rapidly identifies Pseudomonas aeruginosa and distinguishes it from other microorganisms. Rapid testing is important because Pseudomonas aeruginosa is highly resistant to most antibiotics and a faster identification translates into faster suitable treatment.

In summary, the main object of the present invention is to provide a method and kit for identifying Pseudomonas aeruginosa which is fast, reliable, accurate, sensitive, specific and inexpensive. The test of the invention would result in faster treatment of a bacterial infection due to the rapid identification of Pseudomonas aeruginosa by the technique of the present invention.

In accordance with the present invention, a method of identifying a bacteria in a sample, includes providing a sample suspect of comprising a bacteria to be identified, exposing the sample to an antibody specific for a lipoprotein of the bacteria and an agglutination reagent, allowing the sample to react with the antibody and the agglutination reagent, wherein the presence of the bacteria is indicated if an agglutination occurs.

In accordance with the present invention, a kit for testing the presence of a bacteria, such as Pseudomonas aeruginosa, in a sample, includes an agglutination reagent and an antibody specific for a lipoprotein of the bacteria.

### DETAILED DESCRIPTION OF THE INVENTION

The present invention is directed to a method or test (named “Pseudostat II”) for identifying P. aeruginosa from first day cultures with only minimum alternative testing and equipment that is common to most clinical laboratories. The basis for the test is a monoclonal antibody (Mab) that targets lipoprotein I which is unique to P. aeruginosa (De Vos et al., 1993; De Vos et al., 1998; Mutharia et al., 1982; Saint-Onge et al., 1992).

In carrying out the present invention, bacteria were cultivated using standard clinical microbiology procedures, with incubation at 37° C., 5% CO₂, on trypticase-soy agar with 5% sheep blood. Stock quality control strains and some of our immuno-type strains were stored at −70° C. as suspensions in BHI-broth, 20% glycerol. The clinical isolates used in this study were freshly obtained in-house from patients with gram-negative bacterial infections.

Lipoprotein I specific Mab PS2, was prepared by growth of hybridomas in tissue culture flasks using serum and protein free medium (Sigma, St. Louis, Mo.). Mab was purified by ammonium sulfate precipitation followed by dialysis against 0.1 M Trizma. Staphylococcus aureus reagent was prepared by formalin treatment of 24 h cultures of S. aureus (ATCC # 12598, Cowan serotype I), followed by numerous centrifugations and washings in 0.1 M Trizma buffer, pH 7.2, and staining with methylene blue. Extraction and reactivity buffers were prepared using commercially available reagents.

The Pseudostat II test kit included four reagents. Namely, S. aureus, “reagent A”; buffered Mab PS2, “reagent B”; negative control reagent, “reagent C”, and “extraction buffer”. An oxidase spot-test was performed on all 18 and 24 h old isolates. Only oxidase-positive isolates were tested with the Pseudostat II test. All isolates were previously identified using the Vitek gram-negative identification card. Low probability identification calls (<85%) by the Vitek were additionally tested using the API 20 NE test system (bioMerieux Vitek, Inc. Hazelwood, Mo., USA.). Additional tests used to verify isolate identity were: growth on Pseudosel agar (Becton Dickinson Microbiology Systems, Cockeysville, Md., USA), growth at 42° C., and fluorescence under ultraviolet light when grown on Mueller Hinton agar (Kiska and Gilligan, 1999).

The test procedure was performed by removing one or two colonies from agar plates using the cotton tip end of a sterile cotton-tip applicator stick. The swab containing bacteria was immersed into 0.5 ml of extraction buffer (in a 1 ml conical microfuge tube), then twisted and compressed against the side of the tube. The tube was capped and vortexed for 10 sec and then placed in a 37° C. water bath for 5 min. White Time tape, ¾″ width by 1.5″ length, was stuck to an index card for use as the agglutination card. At the end of the extraction, samples were again vortexed for 10 sec. Ten μl of sample were removed with a disposable pipet tip and mixed on the agglutination card with 1 drop of reagent A, and 1 drop of reagent B, then spread in a 2-cm²-diameter oval. The index card with reagents was placed on a lab rotator for 5-8 min. A positive test was recognized as a blue agglutination against a white background. Three controls were run each date of testing. The positive control was P. aeruginosa ATCC #27853. The negative control was Bordetella bronchiseptica ATCC #10580. The reagent control consisted of the Mab suspension buffer without the Mab. A negative test was recognized by the absence of agglutination or agglutination equivalent to that of the reagent control, scored −, or ±, respectfully. Positives were scored as 1+ for granular agglutination, 2+ for flocculate agglutination, 3+ if a string formed, and 4+ if a button formed. Strong 4+ agglutination occurred within 3-5 min whereas weaker reactions took 4-8 min.

A total of 232 P. aeruginosa isolates and 36 other oxidase-positive, gram-negative clinical isolates were tested. Included in the P. aeruginosa isolates were the International Antigenic Type (IAT) strains and 6 mucoid clinical isolates. 75% of the P. aeruginosa isolates including all of the mucoid and IAT strains gave 4+ reactions (Table 1 below). The sensitivity of the test was 96% and the specificity of the test was 91.9% (Table 2 below). Only nine isolates were falsely negative, however one of these, when retested gave a 3+ reaction. In this study, only the first reaction was used with the consideration that if this were being performed in a clinical laboratory, only one test would be performed. The three false-positives, Achromobacter xylosoxidans, Burkholderia cepacia, and Pseudomonas fluorescens, were repeated but consistently gave false-positive reactions. All were re-tested using the API 20 NE system and all gave >95% probabilities for identification. The P. fluorescens isolate was a mucoid environmental isolate that grew at room temperature but not at 37° C. Although it was an exception to study criteria, its inclusion showed that some false-positives occurred if limitations of the test were exceeded.

The reactivity of Mab PS2, has been previously investigated for its reactivity against 470 bacterial isolates using the bio-dot technique (Sciortino, 1993). In that study, Mab PS2 recognized 298/300 P. aeruginosa isolates that included all IAT strains and the Fisher-Devlin immuno-type strains. It did not react with 118/123 other gram-negative bacteria. The outliers in that study to which, Mab PS2 reacted were 3/17 Enterobacter aerogenes, 1/11 Escherichia coli, and 1/2 Proteus vulgaris isolates. For this reason, the use of this test is preferably limited to only oxidase-positive bacteria. Another preference for the test of the invention is that cultures be incubated for at least 18 h. This is because young cultures may not express sufficient amounts of lipoprotein I on the cell surface. For example, isolates that gave 1+ to 3+ reactions when incubated an additional 24 h, all gave 4+ reactions. Some bacteria that were tested showed weak reactivity with the reagent control. This was attributed to the non-specific binding of S. aureus with some component of the digested bacterial mixture. The reagent-control reactivity was subtracted from the isolate-test reactivity for corrected interpretation of reactions.

The following are the details for the method/test of the present invention.

Reagent Preparation

Reagent A (Staph-A co-agglutination reagent). The Cowan strain of Staphyococcus aureus was purchased from the American Type Culture Collection. The reagent was prepared by formalin treatment of 24 h cultures of Staphylococcus aureus followed by numerous washings in buffer and staining with methylene blue.

Staph A Reagent included the following:
Stock reagent of Formalin killed, methylene-blue dyed Staphylococcus aureus in 0.1 M HEPES pH 7.6
500 mM MES buffer, pH 6.0—Suspension buffer
10% FSG (Fish Skin Gelatin)—non-specific blocking reagent
Sodium azide—preservative

Reagent B (Monoclonal antibody). Monoclonal antibody PS2 was purchased commercially. (IMMR, Charleston, S. Carolina. Contact Person: Amy Reid 118 St. Michaels Place, Monks Corner, S.C. 29461)

PS2 Reagent B included the following:
Mab PS2: 3 mg/ml of 0.01 M Trizma buffer, pH 7.6—Stock antibody
500 mM MES buffer, pH 6.0—Stock suspension buffer
0.2 M EDTA [(ethylenedinitrillo)-tetraacetic acid]—Stock chelating agent
10% FSG (Fish Skin Gelatin) in water—Stock non-specific blocking reagent Lysozyme, 10 mg/ml dissolved in 0.01 M glycine/Trizma/NaCl pH 2.5—Stock digestion enzyme
Sodium azide—preservative

Reagent C (Negative control reagent) included the following:
500 mM MES buffer, pH 6.0—Stock suspension buffer
0.2 M EDTA—Stock chelating agent
10% FSG (Fish Skin Gelatin) in water—Stock non-specific blocking reagent
Sodium azide—preservative

Reagent D (Extraction buffer). Extraction and reactivity buffers are prepared in-house using commercially available reagents.

Extraction Buffer included the following:
500 mM MES pH 6.0—Stock suspension buffer
1% Triton×100 in water—Stock suspension detergent
Lysozyme Stock: 10 mg/ml dissolved in 0.01 M glycine/Trizma/NaCl pH 2.5
MgCl₂, 0.1 M —Stock
Sodium azide—preservative
   User Kit

The kit of the invention includes the above-identified three reagents and the negative control reagent. The negative control reagent C, is the same as Reagent B, but lacks the monoclonal antibody. Each reagent is labeled, A, B, C, and Extraction buffer. Preferably, kits contain 100 tests per kit. The kit also contains micro-centrifuge tubes, cotton swabs, tube rack, and time tape with index cards.

### Test Procedure

Preferably, bacterial cultures must be 18 hours old. An oxidase spot-test is performed on suspected colonies. Only oxidase-positive isolates are tested further. The oxidase test is a standard microbiological test performed in all Microbiology laboratories. It requires about two minutes of test time.

Step One

Oxidase-positive colonies are removed from agar plates using a sterile cotton-tip applicator stick. A generous portion of culture us used, i.e., 2-5 colonies. The cotton tip and not the wood end of the applicator stick is used.

Step Two

The cotton swab containing bacteria is immersed into 0.5 ml of extraction buffer (in a microfuge tube).

Step Three

Twist the swab in the tube and wring the swab against the sides of the tube.

Step Four

Cap the tube and place @ 37° C. for 5-10 minutes. (A water bath or heat block is preferable.)

Step Five

Place a white piece of Time tape, preferably ¾″ wide and 1.5″ in length, on an index card.

Step Six

At the end of the extraction period, with the tube capped, briefly vortex the sample. Then mix reagents on the piece of Time tape as follows:
1 drop of Staph A (Reagent A)
1 drop of Antibody reagent (Reagent B)
Add 10 μl of extracted bacterial suspension from the Extraction buffer mix.
   Step Seven

Mix the reagents with the pipet tip, forming an oval circle about the size of a nickel.

Step Eight

Place the index card with reagents on a lab rotator and let rotate for about 5 minutes.

### Interpretation of the Test Results

A positive test is recognized by its agglutination reaction. It is graded, from zero to 4+ reaction. The agglutination reaction is a blue flocculate precipitate against a white background. Some auto-agglutination of reagents may occur for some strains of bacteria. The agglutination seen in the test sample must be greater than the negative control to establish a positive test result. (See the Negative Control procedure below.)

A negative test is recognized by the absence of agglutination.

Negative Control (Performed Simultaneously with the Test Organism Above.)

For each organism tested, a negative reagent control is run as follows:

Step One

Place a white piece of Time tape, preferably ¾″ wide and 1.5″ in length, on an index card next to the piece of tape used for the primary test above.

Step Two

At the end of the extraction period, briefly vortex the reaction tube. Then mix reagents on the piece of Time tape as follows:
1 drop of Staph A (Reagent A)
1 drop of reagent control (Reagent C)

Mix the reagents with the pipet tip, forming an oval circle about the size of a nickel.

Place the index card with reagents on a lab rotator and let it rotate for 5 minutes.

A negative test is recognized by the absence of flocculate agglutination.

Quality Control

Quality control is performed with a strain of Pseudomonas aeruginosa (ATCC #27853) and with Bordetella bronchioseptica ATCC strain, P. aeruginosa (ATCC #27853) gives a positive reaction, whereas B bronchioseptica gives a negative reaction.

From the above, one can readily observe that the Pseudostat II co-agglutination test of the invention would be particularly important to clinical microbiology laboratories for the first day identification of P. aeruginosa. When used in conjunction with colonial morphology, odor, pigmentation, and oxidase-reactivity, it would provide an accurate and rapid means to identify P. aeruginosa and to alert physicians early that P. aeruginosa specific antibiotics should be considered for therapy.

TABLE 1
Reactivity of the Pseudostat II Test for Oxidase-Positive,
Gram-Negative Isolates

| | No. of | | |
|---|---|---|---|
| Organism | Isolates | Reactivity | Interpretation |
| Achromobacter xylosoxidans | 3 | − | Negative |
| subsp. denitrificans | | | |
| Achromobacter xylosoxidans | 1 | 4+ | Positive |
| subsp. xylosoxidans | | | |
| Achromobacter xylosoxidans | 2 | − | Negative |
| subsp. xylosoxidans | | | |
| Alcaligenes odorans | 1 | − | Negative |
| Bordetella bronchiseptica | 2 | − | Negative |
| Burkholderia cepacia | 2 | − | Negative |
| Burkholderia cepacia | 1 | 2+ | Positive |
| *Pseudomonas oryzihabitans | 2 | − | Negative |
| Moraxella catarrhalis | 8 | − | Negative |
| Moraxella lacunata | 1 | − | Negative |
| Pseudomonas aeruginosa | 2 | − | Negative |
| Pseudomonas aeruginosa | 7 | ± | Negative |
| Pseudomonas aeruginosa | 5 | 1+ | Positive |
| Pseudomonas aeruginosa | 20 | 2+ | Positive |
| Pseudomonas aerugiinosa | 23 | 3+ | Positive |
| Pseudomonas aeruginosa | 175 | 4+ | Positive |
| Pseudomonas fluorescens | 1 | − | Negative |
| Pseudomonas fluorescens | 1 | 2+ | Positive |
| Pseudomonas stutzeri | 1 | − | Negative |
| Pseudomonas vesicularis | 1 | − | Negative |
| Pseudomonas putida | 1 | − | Negative |
| Ralstonia pickettii | 3 | − | Negative |
| Ralstonia pickettii | 1 | ± | Negative |
| Shewanella putrefaciens | 1 | − | Negative |
| Shewanella putrefaciens | 1 | ± | Negative |
| Sphingomonas paucimobilis | 3 | − | Negative |

*Weakly oxidase positive isolates
−, negative with no agglutination;
±, negative but equal to the reagent negative control;
1+, positive granular agglutination;
2+ positive flocculate agglutination;
3+, positive string formation with agglutination;
4+, positive agglutination with button formation

TABLE 2
Sensitivity and Specificity of the Pseudostat II Test

| | No. of Isolates | | Sensitivity of | Specificity of |
|---|---|---|---|---|
| Group | Positive | Negative | test (%) | test (%) |
| Pseudomonas | 223 | 9 | 96 | 91.9 |
| aeruginosa | | | | |
| Other gram- | 3 | 34 | | |
| negatives | | | | |

While this invention has been described as having preferred sequences, ranges, steps, materials, or designs, it is understood that it includes further modifications, variations, uses and/or adaptations thereof following in general the principle of the invention, and including such departures from the present disclosure as those come within the known or customary practice in the art to which the invention pertains, and as may be applied to the central features hereinbeforesefforth, and fall within the scope of the invention and of the limits of the appended claims.

The following references, to the extent that they provide exemplary procedural or other details supplementary to those set forth herein, are specifically incorporated herein by reference.

### REFERENCES

Biotec Laboratories Limited. 2002. Biotec In Development. Web site: http://www.biotec.co.uk/development.html. (Apr. 29, 2002.)
BIVDA. 2000. British In Vitro Diagnostics Association. IVD—Healthcare Survey. Web site: http://www.bivda.co.uk/laboratory/index.html. (Apr. 16, 2002.)
Chamberlain, N. 2002. Pseudomonas infections. Kirksville College of Osteopathic Medicine. Web site: httD://www.kcom.edu/facultv/chamberlain/Website/tritzid/pseudinf.htm (Apr. 29, 2002.)
Commonwealth Biotechnologies, Inc. 2002. Web site: http://www.cbi-biotech.com. (Apr. 29, 2002.)
De Vos, D., Bouton, C., Sarniguet, A., De Vos, P., Vauterin, M., and P. Cornelis. 1998. Sequence diversity of the oprI gene, coding for major outer membrane lipoprotein I, among rRNA group I Pseudomonads. J. Bacteriol. 180: 6551-6556.
De Vos, D., Lim, A. Jr., De Vos, P., Sarniguet, A., Kersters, K., and P. Cornelis. 1993. Detection of the outer membrane lipoprotein I and its gene in fluorescent and non-fluorescent pseudomonads: implications for taxonomy and diagnosis. J. Gen. Microbiol. 139:2215-2223.
Dini, L. A., Cockinos, C., Frean, J. A., Niszl, I. A., and M. B. Markus. 2000. Unusual case of Acanthamoeba polyphaga and Pseudomonas aeruginosa keratitis in a contact lens wearer from Gauteng, South Africa. J. Clin. Microbiol. 38:826-829.
Emori, T. G. and R. P. Gaynes. 1993. An overview of nosocomial infections, including the role of the microbiology laboratory. Clin. Microbiol. Rev. 10:428-442.
Finke, M., Duchene, M., Eckhardt, A., Domdey, H., and B. U. von Specht. 1990. Protection against experimental Pseudomonas aeruginosa infection by recombinant P. aeruginosa lipoprotein I expressed in Escherichia coli. Infect. Immun. 58:2241-2244.
Iglewski, B. 2002. Pseudomonas. UTMB Graduate School of Biomedical Sciences. Web site: http://gsbs.utmb.edu/microbook/cho27.htm. (Apr. 29, 2002.)
Jaffe, R. I., J. D. Lane, and C. W. Bates. 2001. Real-time identification of Pseudomonas aeruginosa direct from clinical samples using a rapid extraction method and polymerase chain reaction. Journal of Clinical Laboratory Analysis 15:131-137.
Kiska, D. L., and P. H. Gilligan. 1999. Pseudomonas. In: Murray, P. R., Baron, E. J., Pfaller, M. A., Tenover, F. C., and R. H. Yolken (Eds.), Manual of Clinical Microbiology, 7^{th} ed. American Society for Microbiology, Washington, D.C., USA, pp. 517-525.
Mutharia, L. M, Nicas, T. I., and R. E. W. Hancock. 1982. Outer membrane proteins of Pseudomonas aeruginosa serotype strains. J. Inf. Dis. 146:770-779.
Pollack, M. 2000. Pseudomonas aeruginosa. In: Mandell, G. L., Bennett, J. E., and R. Dolin (Eds.). Principles and practice of infectious diseases, 5^{th} ed. Churchill Livingstone, Philadelphia, Pa., USA, pp. 2310-2335.
Pseudomonas Genome Project. 2002. Pseudomonas aeruginosa Community Annotation Project. Web site: http://www.cmdr.ubc.ca/bobh/PAAP.htm (Apr. 29, 2002.)
Saint-Onge, A., Romeyer, F., Lebel, P., Masson, L. and R. Brousseau. 1992. Specificity of the Pseudomonas aeruginosa PAO1 lipoprotein I gene as a DNA probe and PCR target region within the Pseudomonadaceae. J. Gen. Microbiol. 138:733-341.
Sciortino, C. V. 1993. Surveillance of gram-negative bacteria for Pseudomonas aeruginosa lipoprotein I using a monoclonal antibody. Hybridoma 12:327-332.
Sciortino, C. V., 1993. Surveillance of Gram-negative Bacteria for Pseudomonas aeruginosa Lipoprotein I using a Monoclonal Antibody. Hybridoma, 12 (3):333-342.
Sciortino, C. V., and A. C. Holm. 1987. Monoclonal antibodies to Pseudomonas aeruginosa aminoglycoside-resistance factors. 87th Annual Meeting of the American Society of Microbiology. pp. 34, B56.
Stewart G. S. A. B., Jassim, S. A. A., Denyer, S. P., Newby, P., Linley, K. and Dhir, V. K. 1998. The specific and sensitive detection of bacterial pathogens within 4 hours using bacteriophage amplification. Journal of Applied Microbiology 84, 777-783.
U.S. Food and Drug Administration Center for Devices and Radiological Health. 2002. An Introduction to Medical Device Regulations (FDA 924222)
Washington State University. Biology Bulletin Board. Pseudomonas. 2002. Web site: http://www.sci.wsu.edu/bio/micro310pseudomonas.html (Apr. 29, 2002.)

1. A method of identifying a bacteria in a sample, comprising the steps of:
a) providing a sample suspect of comprising a bacteria to be identified;
b) exposing the sample to an antibody specific for a lipoprotein of the bacteria and an agglutination reagent;
c) allowing the sample to react with the antibody and the agglutination reagent; and
d) whereby the presence of the bacteria is indicated if an agglutination occurs.

2. The method of claim 1, wherein:
the bacteria to be identified comprises an oxidase-positive organism.

3. The method of claim 2, wherein:
the antibody comprises monoclonal antibody PS2.

4. The method of claim 3, wherein:
the agglutination reagent comprises a strain of Staphylococcus bacteria.

5. The method of claim 4, wherein:
the organism comprises Pseudomonas aeruginosa.

6. A method of identifying Pseudomonas aeruginosa in a sample, comprising the steps of:
a) providing a sample suspect of comprising Pseudomonas aeruginosa;
b) cultivating organism in a suitable medium;
c) removing a portion of the cultured organism and exposing to a first reagent;
d) exposing the organism obtained in step c) to second and third reagents, the second reagent comprising an agglutination reagent and the third reagent comprising an antibody specific for a lipoprotein of Pseudomonas aeruginosa;
e) allowing the components in step d) to react; and
f) whereby the presence of Pseudomonas aeruginosa is indicated if an agglutination occurs.

7. The method of claim 6, wherein:
the step c) of removing and exposing a portion of the cultured organism to a first reagent comprises extracting from the Pseudomonas aeruginosa organism a lipoprotein by an extraction reagent.

8. The method of claim 7, wherein:
the lipoprotein comprises lipoprotein 1.

9. The method of claim 6, wherein:
the agglutination reagent comprises a strain of Staphylococcus bacteria.

10. The method of claim 9, wherein:
the Staphylococcus strain is stained generally blue; and
the presence of Pseudomonas aeruginosa is indicated by a generally blue agglutination.

11. The method of claim 9, wherein:
the antibody comprises monoclonal antibody PS2.

12. The method of claim 6, wherein:
the step b) comprises incubating for a period of about 18 to 24 hours.

13. The method of claim 6, further comprising the step of:
g) isolating oxidase-positive organism form the medium prior to step c).

14. A kit for testing the presence of Pseudomonas aeruginosa in a sample, comprising:
a) an agglutination reagent; and
b) an antibody specific for a lipoprotein of Pseudomonas aeruginosa.

15. The kit of claim 14, further comprising:
a) a reagent for extracting the lipoprotein from Pseudomonas aeruginosa.

16. The kit of claim 15, wherein:
a) the agglutination reagent comprises a strain of Staphylococcus bacteria.

17. The kit of claim 16, wherein:
a) the antibody comprises monoclonal antibody PS2.

18. The kit of claim 15, further comprising:
a) a negative control reagent.
